(19) United States
(12) Patent Application Publication
(10) Pub. No.: US 2004/0166062 A1
(43) Pub. Date: Aug. 26, 2004

(54) Ablation therapy using chemical shift magnetic resonance imaging

(76) Inventors: David A. Roberts, Bryn Mawr, PA (US); Mark A. Rosen, Bala Cynwyd, PA (US); John S. Leigh JR., Philadelphia, PA (US)

Correspondence Address: NATH & ASSOCIATES PLLC, Sixth Floor, 1030 15th Street, N.W., Washington, DC, 20005, US

(21) Appl. No.: 10/369,775

(22) Filed: Feb. 21, 2003

(51) Int. Cl. A61K 49/00

(52) U.S. Cl. 424/9.3

(57) ABSTRACT

The present invention relates to novel methods for direct visualization of the distribution of therapeutic agents, such as acetic acid, in the tissue of an animal, utilizing chemical shift magnetic resonance imaging (MRI). Said methods are particularly useful for percutaneous chemical ablation procedures to provide an optimal dosage of chemical ablation agent such as acetic acid to target tissues such as tumors, and for limiting damage to surrounding tissues.

This work was supported in part by National Institutes of Health grant RR02305. The United States government may have rights in this invention by virtue of this support.

### BACKGROUND OF THE INVENTION

1. Field of Invention

The present invention relates to novel methods for direct visualization of the distribution of therapeutic agents, such as acetic acid, in the tissue of an animal, utilizing chemical shift magnetic resonance imaging (MRI). Said methods are particularly useful for percutaneous chemical ablation procedures to provide an optimal dosage of chemical ablation agent such as acetic acid to target tissues such as tumors, and for limiting damage to surrounding tissues.

2. Background

U.S. Pat. No. 4,687,658, issued Aug. 18, 1987 to Quay, discloses the use of homologs of Diester-DTPA-Paramagnetic compounds (such as dimethyl acetyl diethylene triamine triacetic acid) as contrast agents for magnetic resonance imaging (MRI).

U.S. Pat. No. 5,799,059, issued Aug. 25, 1998 to Stembridge, et al., discloses a transparent phantom apparatus for monitoring patient support table movements during computer assisted tomography (CAT) and magnetic resonance imaging (MRI). The phantom is made of a hollow straight tube of transparent material filled with air or a liquid, wherein for CAT scan systems, the liquid may optionally have distilled water with a few drops of weak acetic acid as an algicide, and for MRI the liquid may optionally have a contrast enhancer which is preferably a copper sulfate solution.

Carpenter T A, Hall L D, and Hogan P G, Magnetic Resonance Imaging of the Delivery of a Paramagnetic Contrast Agent by an Osmotic Pump, Drug Des Deliv 3(3):263-6 (1988) disclose the use of Magnetic Resonance Imaging (MRI) in verifying the in vitro delivery action of an osmotically-driven pump, employing gadolinium diethylene triamine penta acetic acid as a contrast agent to overcome the inability of MRI to detect drugs commonly used with such pumps. See, also, Carr D H and Gadian D G, Contrast Agents in Magnetic Resonance Imaging, Clin Radiol 36(6) :561-8 (1985), who also disclose gadolinium diethylene triamine penta acetic acid as an effective paramagnetic contrast agent in MRI.

However, each of these MRI technologies is based upon the use of at least two independent agents or techniques, at least one, a contrast agent, during the MRI screening and at least one more, a treatment agent or technique, during the actual treatment of a target disease, disorder, or condition. The present inventive subject matter addresses the need for a single agent which serves both a therapeutic function and acts as a contrast agent for MRI, resulting in greatly improved targeting of the therapeutic agent to target tissues.

Liver cancer is a significant cause of mortality and morbidity in the United States. In 1999, there were 14,500 new cases of primary hepatocellular carcinoma and 13,600 deaths attributable to this tumor. Despite the decreasing incidences of many other solid tumors in the United States, the incidence of hepatocellular carcinoma continues to increase, largely in parallel to an increasing incidence of viral hepatitis.

There are approximately 4 million people in the United States who are currently infected with the hepatitis C virus (HCV), and of the chronic HCV carriers, up to 20% will develop cirrhosis of the liver, in turn resulting in 2-7% who will develop liver cancer within 20 years of infection. Although in many cases surgical resection or transplantation offers the only chance for cure, most patients with hepatocellular carcinoma are not candidates for these treatments. Recently, several minimally invasive, nonsurgical local therapies have been described. These include radio-frequency ablation, laser-induced thermotherapy, and chemical ablation using hot saline, ethanol, and/or acetic acid. When performed for hepatocellular carcinoma less than about 3 cm, chemical ablation can achieve 5-year survival rates of 60%, equivalent to those of surgical resection. A randomized, controlled trial has suggested that acetic acid ablation is more effective than ethanol ablation in patients with hepatocellular carcinoma less than about 3 cm. Chemical ablation has most commonly been performed using either ultrasound or computer-assisted tomography (CT) guidance.

Unfortunately, these modalities do not allow for direct visualization of the chemical agents being injected. Instead, a volume of the chemical agent is injected that is determined based on the morphology and apparent size of the lesion as determined by external examination and imaging.

Applicants have solved the problem of direct visualization by utilizing chemical shift MRI to detect the injection of a therapeutic composition, such as acetic acid, into a percutaneous tumor. Unexpectedly, Applicants have found that the distribution of a therapeutic composition, such as acetic acid, may be directly visualized in the tissue of an animal utilizing chemical shift MRI. Phantom data and ex vivo data, as discussed below, demonstrate focal increases in the observed signal in chemical shift MRI that correlate well with the site of injection, and also show the undesired spread of the agent into a vascular space. This unexpected result permits direct visualization of the distribution of the therapeutic agent in percutaneous chemical ablation procedures, allows optimization of the chemical ablation agent dosage, and thereby permits both optimal targeting of tissues such as tumors and less damage to surrounding tissues. In a preferred embodiment of the present invention, the direct visualization of the distribution of the therapeutic agent is made both in terms of the absolute distribution of the therapeutic agent and in terms of the relative concentration of the therapeutic agent using spatially-localized spectroscopy techniques.

### SUMMARY OF THE INVENTION

The present invention relates to a method for mapping distribution of a therapeutic composition in the tissue of an animal, which comprises the steps of:

(i) administering said therapeutic composition to the tissue of said animal, and

(ii) directly visualizing the distribution of said therapeutic composition by chemical shift magnetic resonance imaging attuned to detect said therapeutic composition.

The present invention further relates to a method for mapping distribution of an acetic acid composition during a chemical ablation therapy procedure in a tumor in an animal, which comprises the steps of:

(i) injecting said acetic acid composition into said tumor in an animal, and

(ii) directly visualizing the distribution of said composition by chemical shift magnetic resonance imaging attuned to detect acetic acid resonance.

The present invention further relates to a method for treating an animal in need of chemical ablation therapy in a target tissue, comprising the steps of:

(i) injecting a therapeutic composition into said target tissue of said animal; and

(ii) directly visualizing the distribution of said therapeutic composition using chemical shift magnetic resonance imaging attuned to detect the resonance of said therapeutic composition.

### BRIEF DESCRIPTION OF THE DRAWINGS

FIGS. 1A, 1B, and 1C are graphs which depict the Proton NMR spectrum of water, 80% acetic acid, and absolute ethanol.

FIG. 2 is a photograph which depicts a series of CS-MRI images of phantoms consisting of vials of glacial acetic acid, absolute ethanol, and water.

FIG. 3 is a series of photographs which depict changes in CS-MRI imaging signal obtained using a selective suppression pulse sequence during the slow injection of glacial acetic acid.

FIGS. 4A, 4B, 4C, and 4D are a series of photographs which depict the time-course of treatment of patient 1 with acetic acid ablation therapy.

FIGS. 5A, 5B, 5C, and 5D are a series of photographs which depict the time-course of treatment of patient 2 with acetic acid ablation therapy.

FIGS. 6A and 6B are a series of photographs which depict the time-course of absolute ethanol ablation therapy of a hepatoma in patient 3.

### DETAILED DESCRIPTION OF THE INVENTION

### Definitions

The term “magnetic resonance imaging” or “MRI” as used herein refers to a non-invasive imaging technique which detects atomic resonance from one or more atom(s), or small groups of atoms, having particular proton resonance characteristics when excited by electromagnetic energy at a resonance frequency of the atom, in the presence of one or more magnetic field(s). Resonance is determined at a large number of points throughout the target tissue and assembled by detection instrument(s) into a two- or three-dimensional image map depicting the characteristics of the target tissues.

The term “chemical shift magnetic resonance imaging” as used herein refers to the MRI technique in which the detection instrument(s) is/are attuned to detect a particular resonance frequency which is different than the resonance frequency of ¹H.

The term “resonance” as used herein refers to the process of absorption and emission of electromagnetic energy by protons in the nucleus of an atom, at the resonance frequency of the atom. The resonance frequency of an atom is determined by the strength of the applied magnetic field(s) and the microenvironment of the atom.

The term “contrast agent” as used herein refers to a composition administered to a patient undergoing an MRI in order to improve the contrast or resolution between tissues. An MRI contrast agent predictably alters the local magnetic field in the tissue being examined, accentuating some resonance differences and diminishing others.

The term “attuned” as used herein refers to selection of magnetic resonance imaging parameters in order to maximize the resonance signal of a particular chemical molecule or moiety.

The term “¹³C-labeled” as used herein refers to compounds having one or more carbon atoms replaced by ¹³C.

The term “²³Na-labeled” as used herein refers to compounds having one or more sodium atoms replaced by ²³Na.

The term “direct visualization” as used herein refers to the real time visualization of a circumstance or event using an imaging means which provides an essentially instant depiction of the imaged circumstance or event. The imaging means contemplated in this application is selected from the group consisting of NMR, MRI, X-ray, Gamma-ray, fluorescent, and , but most preferably is MRI.

The term “chemical ablation therapy” as used herein refers to the process of injecting or otherwise treating tissue with a chemical composition which produces cell death and/or tissue necrosis in the target tissue.

The term “percutaneous” as used herein refers to a route of administration through the skin of an animal.

The term “phantom” as used herein refers to a simulation which takes the place of actual tissue in an experimental procedure, such as the use of a plastic bag or vial containing the target compound in an MRI procedure.

### Methods of the Present Invention

The use of existing MRI technology in a treatment is based upon the use of at least two independent agents or techniques: at least one agent, a contrast agent, is used during the MRI screening and at least one more agent or technique, a treatment agent or technique, is used during the actual treatment of a target disease, disorder, or condition. Unfortunately, this procedure does not allow for direct visualization of the treatment agent being injected or the effect of the technique being used.

In order to address the need for direct visualization of chemical agents as they are injected, we have developed novel methods for direct visualization of the distribution of chemical compositions in the tissue of an animal, utilizing chemical shift magnetic resonance imaging. The ability to detect and visualize the therapeutic composition is unexpected, resulting in an unprecedented, simple, and practical means for optimizing treatments such as chemical ablation therapy, and monitoring such treatments with a greater degree of accuracy than heretofore possible.

Thus, the present invention relates to a method for mapping distribution of a therapeutic composition in the tissue of an animal, which comprises the steps of:

(i) administering said therapeutic composition to the tissue of said animal, and

(ii) directly visualizing the distribution of said therapeutic composition by chemical shift magnetic resonance imaging attuned to detect said therapeutic composition.

In a preferred embodiment, said therapeutic composition is selected from the group consisting of acetic acid, ethanol, ¹³C-labeled acetic acid, ¹³C-labeled ethanol, and ²³Na-labeled saline.

In a more preferred embodiment, said therapeutic composition is acetic acid.

In another preferred embodiment, said magnetic resonance imaging utilizes acetic acid as a contrast agent and is attuned to detect acetic acid resonance.

In another preferred embodiment, said direct visualization is made during administration of said therapeutic composition in a chemical ablation therapy procedure.

In another preferred embodiment, said administration is made by injecting said therapeutic composition into said animal through one or more injection site(s).

In another preferred embodiment, said tissue is a tumor.

In a further preferred embodiment, said tumor is a hepatic tumor, a renal tumor, a prostate tumor, a lung tumor, or a breast tumor.

In another preferred embodiment, said method additionally comprises a first step of injecting said tissue with a contrast agent before administering said therapeutic composition and before visualizing the distribution of said therapeutic composition.

In a more preferred embodiment, said contrast agent is a gadolinium-containing contrast agent.

It is expected that by injecting a contrast agent before the therapeutic composition, the contrast agent will help better define the boundaries of therapeutic agent in the target tissue, and thus improve the targeting of an effective dose of the therapeutic composition to the target tissues, and reduce the amount of damage to surrounding, non-target tissues.

In a further preferred embodiment, said step of directly visualizing the distribution of said therapeutic composition additionally comprises scanning over a range of chemical shift magnetic resonances attuned to different concentrations of said therapeutic composition.

Scanning over a range of chemical shift magnetic resonances attuned to different concentrations of said herapeutic composition will permit determination of the relative concentration of the therapeutic composition in three dimensional space. It is expected that determination of the relative concentration of the therapeutic composition in three dimensional space will permit more precise targeting of tissues with an effective amount of the therapeutic composition, and thus produce even less damage to surrounding tissues. Thus, in a most preferred embodiment of the present invention, the direct visualization of the distribution of the therapeutic agent is made both in terms of the absolute distribution of the therapeutic agent and in terms of the relative concentration of the therapeutic agent using spatially-localized spectroscopy techniques.

The present invention further relates to a method for mapping distribution of an acetic acid composition during a chemical ablation therapy procedure in a tumor in an animal, which comprises the steps of:

(i) injecting said acetic acid composition into said tumor in an animal, and

(ii) directly visualizing the distribution of said composition by chemical shift magnetic resonance imaging attuned to detect acetic acid resonance.

In a preferred embodiment, said method additionally comprises a first step of injecting said tissue with a contrast agent before administering said therapeutic composition and before visualizing the distribution of said therapeutic composition.

In a more preferred embodiment, said contrast agent is a gadolinium-containing contrast agent.

In a further preferred embodiment, said step of directly visualizing the distribution of said composition additionally comprises scanning over a range of chemical shift magnetic resonances attuned to different concentrations of said composition.

The present invention further relates to a method for treating an animal in need of chemical ablation therapy in target tissue, comprising the steps of:

(i) injecting a therapeutic composition into said target tissue of said animal; and

(ii) directly visualizing the distribution of said therapeutic composition using chemical shift magnetic resonance imaging attuned to detect the resonance of said therapeutic composition.

In a preferred embodiment, said method comprises the additional step of selecting the volume of said therapeutic composition to be injected, and optionally selecting one or more additional target tissue injection site(s), based on the distribution of said therapeutic composition visualized.

In another preferred embodiment, said therapeutic composition is acetic acid.

In a preferred embodiment, said target tissue is a tumor.

In a further preferred embodiment, said tumor is a hepatic tumor, a renal tumor, a prostate tumor, a lung tumor, or a breast tumor.

In another preferred embodiment, said method additionally comprises a first step of injecting said tissue with a contrast agent before administering said therapeutic composition and before visualizing the distribution of said therapeutic composition.

In a more preferred embodiment, said contrast agent is a gadolinium-containing contrast agent.

In another preferred embodiment, said step of directly visualizing the distribution of said therapeutic composition additionally comprises scanning over a range of chemical shift magnetic resonances attuned to different concentrations of said therapeutic composition.

Magnetic Resonance Imaging (MRI). When protons are placed in a magnetic field, they become capable of receiving and then transmitting electromagnetic energy. The strength of the transmitted energy is proportional to the number of protons in the tissue. Signal strength is modified by properties of each proton's microenvironment, such as its mobility and the local homogeneity of the magnetic field. MR signal can be weighted to accentuate some properties and not others.

When an additional magnetic field, one which is carefully varied in strength at different points in space, is superimposed over the first, each point in space has a unique radio frequency at which the signal is received and transmitted. This makes constructing an image possible.

MR signal sources. In a magnetic field, protons oscillate at a frequency which depends on the strength of the magnetic field. Protons are capable of absorbing energy if exposed to electromagnetic energy at the frequency of oscillation. After they absorb energy, the nuclei re-radiate this energy and return to their equilibrium state. This re-radiation or transmission of energy by the nuclei as they return to their initial state is the observed MRI signal, which takes place over a short but measurable period of time.

The return of nuclei to their equilibrium state is governed by two physical processes, and time that it takes for these two relaxation processes to take place is roughly equal to:

1. the relaxation back to equilibrium of the component of the nuclear magnetization which is parallel to the magnetic field, time T1, and

2. the relaxation back to equilibrium of the component of the nuclear magnetization which is perpendicular to the magnetic field, time T2.

The strength of the MRI signal depends primarily on three parameters:

1. Density of protons in a tissue: the greater the density of protons, the larger the signal will be,

2. T1, and

3. T2.

The contrast between tissues is dependent upon how these 3 parameters differ between tissues. For most “soft” tissues in the body, the proton density is very homogeneous and therefore does not contribute in a major way to signal differences seen in a image. However, T1 and T2 can be dramatically different for different soft tissues, and these parameters are responsible for the major contrast between soft tissues.

T1 and T2 are strongly influenced by the viscosity or rigidity of a tissue. Generally speaking, the greater the viscosity and rigidity, the smaller the value for T1 and T2. It is possible to manipulate the MR signal by changing the way in which the nuclei are initially subjected to electromagnetic energy. This manipulation can change the dependence of the observed signal on the three parameters: proton density, T1 and T2. Hence, one has a number of different MR imaging weightings to choose from, which accentuate some properties and not others.

Basic Proton MR Imaging Theory. In the nucleus of every atom, individual protons and neutrons spin about an axis. This property, called spin angular momentum, is the basis of nuclear magnetism. Since atomic nuclei have charge, this spinning motion produces a magnetic moment along the spin axis. In most nuclei, the particles are paired so that the net magnetic properties cancel. However, if the number of protons or neutrons is odd, complete cancellation is not possible. Nuclei with an unpaired proton or neutron such as hydrogen 1, carbon 13, and sodium 23, among others, exhibit a net magnetic effect. The relative strength of this magnetic moment is a property of the type of nucleus and therefore determines the MR detection sensitivity. The hydrogen (¹H) nucleus, which is highly abundant in biological systems, has the strongest magnetic moment. It is ideal for MRI because its nucleus as a single proton and a large magnetic moment. The large magnetic moment means that, when placed in a magnetic field, the hydrogen atom has a strong tendency to line up with the direction of the magnetic field.

Since the individual magnetic moments or axes of spin are randomly oriented, biological tissue does not normally exhibit a net magnetization. However, in the presence of an external static magnetic field, the individual magnetic moments tend to align either parallel or antiparallel to the direction of the applied field. The magnets in use today in medical imaging MRI are in the 0.5-tesla to 2.0-tesla range, or 5,000 to 20,000 gauss. Magnetic fields up to 60 Tesla are used in research.

Since a parallel alignment to the field is the lower energy state and thus the preferred energy state, slightly more nuclei will align parallel rather than antiparallel to the field. As a result, a tissue will exhibit a net magnetization. The individual spins do not align exactly parallel to the applied field, but at an angle to it. The individual spins cause the moment to precess about the magnetic axis. The frequency with which the moment precesses is given by the Larmor equation: ζB₀=f, where B₀=strength of the applied magnetic field; ζ=gyromagnetic ratio, which is related to the strength of the magnetic moment for the type of nuclei; and f=the frequency of precession, the Larmor frequency. For example, for the hydrogen atom ζ=4257 Hz/Gauss. Therefore, at B₀=1.5 Tesla, the Larmor frequency is 63.855 MHz.

In order to create an MR signal which can be detected, a resonance condition, an alternating absorption and dissipation of energy, must be established. In the external static magnetic field, nuclei can be shifted from the parallel to antiparallel alignment by the application of radio frequency energy. Application of radio frequency (“RF”) magnetic field at the Larmor frequency results in energy absorption, while RF energy applied at other frequencies has no effect. If we consider a secondary or gradient RF magnetic field applied perpendicular to the primary magnetic field, the system will absorb energy and begin to precess about the primary magnetic field axis. These secondary magnets are very low strength compared to the main magnetic field; they may range in strength from 180 gauss to 270 gauss, or 18 to 27 millitesla. The function of the gradient magnets is to create a variable secondary magnetic field.

If the RF energy is pulsed, the net magnetization is rotated at an angle away from the primary magnetic field axis. This angle is the flip angle and is proportional to the duration and amplitude of the RF pulse. Upon termination of the RF pulse, the nuclei return to their original alignment parallel to the applied static field and energy is emitted in the form of an RF signal. The frequency of the emitted signal depends on the strength of the applied static magnetic field as well as the type of nuclei producing the signal. The MRI machine applies an RF pulse that is specific to the target nuclei. Detection and analysis of this signal provide insight into the chemical composition of the material. This process of alternating absorption and emission of RF energy by the material is termed magnetic resonance (MR).

At the end of the applied RF pulse, the RF signal emitted by the material is at its maximum intensity. The signal intensity diminishes rapidly within a few hundred milliseconds as the higher, antiparallel, energy state is depopulated and the nuclei return to their original energy state. This RF signal is picked up by a receiver coil. The waveform of this signal is an exponentially damped sine wave and is called the free induction decay.

In order to produce an image, each MR signal must be referenced to a specific region of tissue. This is accomplished by applying a gradient magnetic field in which the field strength varies linearly with position. The gradient gradually varies the magnetic field strength resulting in a corresponding shift in the RF frequency needed to stimulate the tissue. Since emitted RF signals will also demonstrate a shift in frequency, the excited tissue from which the signals originated can be localized. Using a computer-aided reconstruction program, similar to that used in computed tomography, the signals attributed to individual volume elements of tissue can be resolved and reconstructed into an image. The most common method of image reconstruction is the two-dimensional Fourier transform.

The addition of contrast agents in many cases improves MRI sensitivity and/or specificity. Traditional MRI contrast materials work by altering the local magnetic field in the tissue being examined. There are essentially four types of traditional MRI contrast materials: diamagnetic, paramagnetic, superparamagnetic, and ferromagnetic.

Diamagnetic materials have no intrinsic atomic magnetic moment, but when placed in a magnetic field weakly repel the field, resulting in a small negative magnetic susceptibility. Materials like water, copper, nitrogen, barium sulfate, and most tissues are diamagnetic.

Superparamagnetic materials consist of individual domains of elements that have ferromagnetic properties in bulk. Their magnetic susceptibility is between that of ferromagnetic and paramagnetic materials. Examples of a superparamagnetic materials include iron containing contrast agents for bowel, liver, and lymph node imaging.

Paramagnetic materials include oxygen and ions of various metals like Fe, Mg, and Gd. These ions have unpaired electrons, resulting in a positive magnetic susceptibility. The magnitude of this susceptibility is less than one one-thousands of that of ferromagnetic materials. The effect on MRI is increase in the T1 and T2 relaxation rates (decrease in the T1 and T2 times). Gd is commonly used in MR contrast agents. At the proper concentration, Gd contrast agents cause preferential T1 relaxation enhancement, causing increase in signal on T1-weighted images. At high concentrations, loss of signal is seen instead as a result of the T2 relaxation effects dominating.

Ferromagnetic materials generally contain iron, nickel, or cobalt. These materials have a large positive magnetic susceptibility, i.e., when placed in a magnet field, the field strength is much stronger inside the material than outside. The ability to remain magnetized when an external magnetic field is removed is a distinguishing factor compared to paramagnetic, superparamagnetic, and diamagnetic materials. On MR images, these materials may cause susceptibility artifacts characterized by loss of signal and spatial distortion.

Chemical shift MRI is effectively the selective imaging of subset of resonances within an NMR spectrum. Several methods have previously been described for the performance of CS-MRI, including selective excitation, selective suppression, and in/out-of-phase gradient-echo MRI. In the selective excitation (“SE”) approach, an RF pulse that is both spatially and spectrally selective is used to excite only the desired spectral components in a defined region in space, typically a two-dimensional slice. In the selective suppression (“SS”) approach, an off-resonance RF pulse is used to selectively suppress the signal arising from resonances at a fixed frequency offset from the imaged resonance. The SS method is the most common method of chemical fat suppression used in clinical imaging. However, it may equally well be applied to perform water suppression. In the in/out-of-phase (“I/O”) MRI method, gradient-echo images are acquired using two different echo times such that two resonances that differ in frequency are in-phase in one acquisition, and 180 degrees out of phase in the other acquisition. Subtraction of the echo profiles then provides a selective image of the shifted resonance, effectively suppressing other signal contributions.

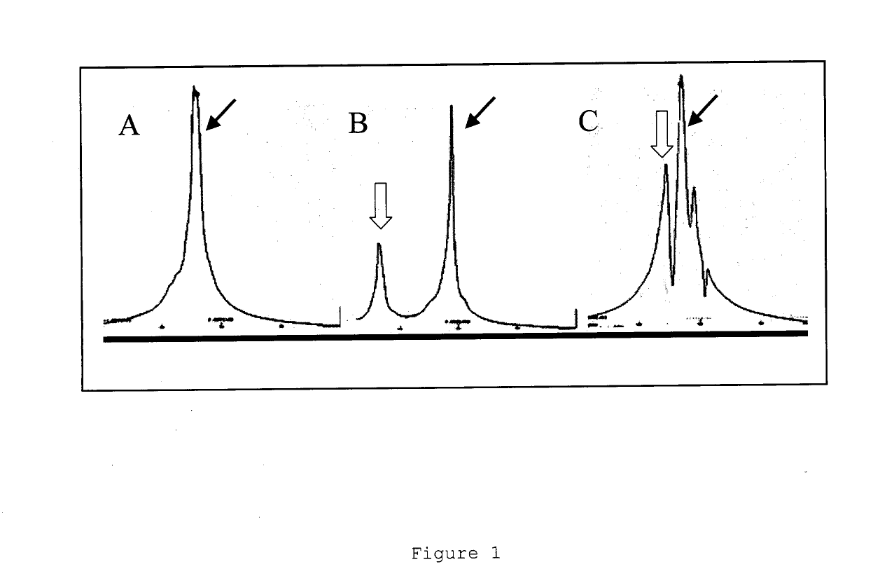

We have overcome the necessity for contrast agents and have developed novel methods for direct visualization of the distribution of chemical compositions in the tissue of an animal, utilizing chemical shift magnetic resonance imaging, “CS-MRI”. The NMR spectrum of acetic acid and ethanol appear in FIG. 1. The NMR-visible signal of acetic acid is principally derived from the protons of the methyl group of the acetate component of the molecule. These protons resonate at a chemical shift of approximately −2.7 ppm, or −170 Hz away from the water resonance at 1.5 Tesla. This chemical shift of −170 Hz is similar to that of lipid in the human body, which is approximately −220 Hz at 1.5 Tesla. The spectrum of ethanol is more complex as a result of splitting of peaks by multiple chemically distinct protons on the methyl and ethylene groups. However, the results depicted in FIG. 1 demonstrate that the spectrum contains a significant energy resonance at a band of frequencies that are specific for the ethanol molecule, frequencies that are distinct from the central water resonance and ranging from approximately 50 to 200 Hz below that of the water proton resonance, at 1.5 Tesla.

Chemical shift MRI has been used in numerous clinical applications, including the evaluation of adrenal masses. Unexpectedly, we have found that chemical shift MRI may be used to obtain real-time images of the distribution of an active agent, such as acetic acid or ethanol, during percutaneous procedures, such as chemical ablation therapy, involving the injection of the active agent into the tissues of an animal.

We expect that results similar to those found for acetic acid and ethanol are attainable using ¹³C-labeled acetic acid, ¹³C-labeled ethanol, and hot ²³Na-labeled saline. Each is an effective chemical ablation composition and has one or more unpaired proton(s) for MRI detection. We expect that the chemical shift of the methyl group protons of ethanol, the ¹³C proton of ¹³C-labeled acetic acid or ethanol, or the ²³Na proton of ²³Na-labeled saline may be identified and utilized to directly visualize each of those compositions using chemical shift MRI.

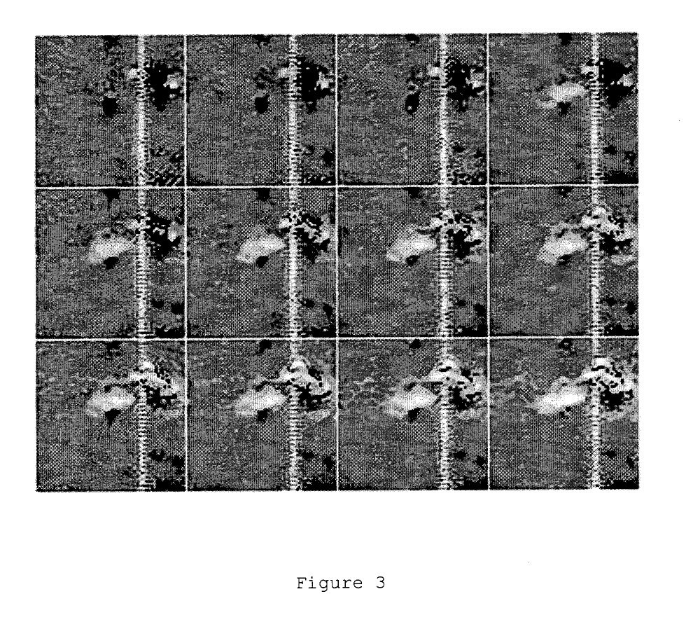

These methods are expected to improve the results obtained using acetic acid, and other, ablation therapy in patients under treatment for liver cancer. There are several risks of chemical ablation, including capsular perforation, tract seeding, nephrotoxicity, and intraperitoneal hemorrhage. Chemical shift MRI allows the radiologist to tailor the volume of acetic acid to the size of the tumor being treated. Chemical shift MRI also provides a direct means to detect the undesired spread of the agent into adjacent structures, as occurred during the ex vivo testing (FIG. 3), and also aids in defining the relationship of the agent with the liver capsule. It is expected that the qualitative distribution of the agent identified on chemical shift MRI correlates with areas of tumor necrosis and clinical response. These results suggest that chemical shift MRI will in the future help to improve the safety and efficacy of percutaneous chemical ablation.

### EXAMPLES

The following examples are illustrative of the present invention and are not intended to be limitations thereon. Unless otherwise indicated, all percentages are based upon 100% by weight of the final composition.

### Example 1

### Determination of Acetate and Ethanol Resonance Shift

The following example illustrates determination of the acetate resonance shift for acetic acid and for ethanol. Saline control, acetic acid, and ethanol phantoms were constructed using a 500 mL bag of normal saline, a 100 mL plastic vial filled with 100%, or ‘glacial’, acetic acid, and a 100 mL plastic vial filled with 100% ethanol. The phantoms were placed in a 10 cm birdcage transmit/receive coil on a 1.5 Tesla MRI system. Transverse images of the phantoms were then acquired using a two-dimensional, spoiled, gradient-echo pulse sequence (SPGR) that incorporated an off-resonance chemical saturation RF pulse. Images of the phantoms were generated with the spectrometer frequency centered on the acetate resonance which was determined to be shifted −170 Hz from the water resonance, as shown in FIG. 1. The offset frequency of the chemical shift suppression pulse was set at 170 Hz. The scanning parameters were: 30 degree flip angle, TE=4.2 msec, TR=34 msec, 24 cm field-of-view 16 kHz bandwidth, 256×128 matrix, and 8 mm slice thickness.

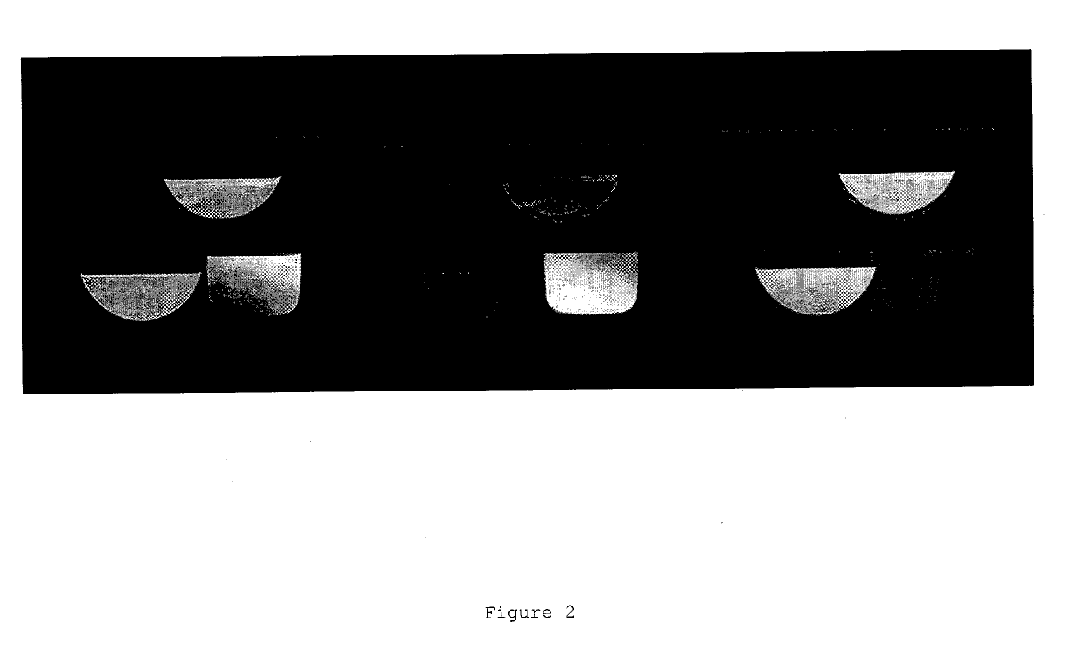

Images of the phantoms, consisting of vials of glacial acetic acid (AA), absolute ethanol (E), and water (W), obtained using the a suppressed pulse sequence appear in FIG. 2. In the left image, obtained without an RF suppression pulse, shows all three. In the middle image, the water-suppressed sequence demonstrates a selective image with signal only coming from the vials of acetic acid and ethanol. No water signal is present. In the right image, the acetic acid/ethanol-suppressed sequence demonstrates a selective image with signal only coming from the vial of water. No acetic acid or ethanol signal is present. These data demonstrate the effectiveness of Applicants' methods in creating selective images of the acetic acid and ethanol resonance(s).

### Example 2

### Ex Vivo Imaging of Acetic Acid Injection

The following example illustrates an ex vivo study of images obtained during the slow injection of glacial acetic acid using the methods of the present invention. An MRI-compatible, 10 cm, 22-gauge needle was inserted into a calf liver that was placed in a transmit/receive birdcage coil. After acquisition of baseline proton images, a water-suppressed, three-dimensional, compact gradient-echo pulse sequence was used to obtain images during the slow injection of glacial acetic acid. The spectrometer frequency was centered on the acetic acid resonance. The offset frequency of the chemical saturation pulse was set at 170 Hz. 8 cc of glacial acetic acid was injected slowly by hand over 2 minutes following the acquisition of baseline images. A complete 3D data set was acquired every 15 seconds. Scanning parameters were: 20 degree flip angle, TE=4.2 msec, TR=34 msec, matrix=256×128×4, 1 signal average, and 32 kHz receiver bandwidth. A total of 15 temporal phases was acquired, each with approximately 15 second time resolution.

FIG. 3 shows ex vivo data obtained in a calf liver during slow injection of acetic acid via an MRI-compatible needle. The baseline acquisition (time 0 seconds) shows an interference band resulting from ambient radio-frequency noise (horizontal arrow). Subsequent images show focal accumulation of acetic acid adjacent to the needle tip (vertical arrows). Also seen on later images is focal accumulation in a blood vessel adjacent to the site of injection (thin arrows), providing a direct means to detect the undesired spread of the agent into adjacent structures. Extravasation of the agent into a large portal vein branch is observed (arrows) on later frames. The time after injection appears in seconds in the lower left corner of each frame.

### Example 3

### In Vivo Imaging of Acetic Acid Injection

The following example illustrates an in vivo study of images obtained during the slow injection of glacial acetic acid using the methods of the present invention. Two human patients with unresectable hepatocellular carcinoma were referred for acetic acid ablation therapy. Each patient had undergone a standard liver MRI examination including dynamic gadolinium injection several days prior to the performance of the ablation study.

Each patient was placed supine on the MRI scanner platform and a vial of dilute (25%) acetic acid was taped to the anterior abdominal wall to allow for spectrometer calibration. After obtaining standard T1 and T2-weighted imaging, an MR-compatible 10 cm long, 22-gauge needle was advanced into the lesions. Baseline imaging was performed using a water-suppressed two-dimensional spoiled gradient-echo pulse sequence. The spectrometer frequency was then centered during a manual calibration on the acetic acid peak, and the frequency offset of the chemical saturation pulse was set at 170 Hz, as above. Dynamic two-dimensional, chemical shift MRI was then performed during the slow injection of acetic acid. The scanning parameters were: 30 degree flip angle, TE=4.2 msec, TR=34 msec, 34-38 cm field-of-view, ±16 kHz receiver bandwidth, 256×128 matrix, and 7 mm slice thickness.

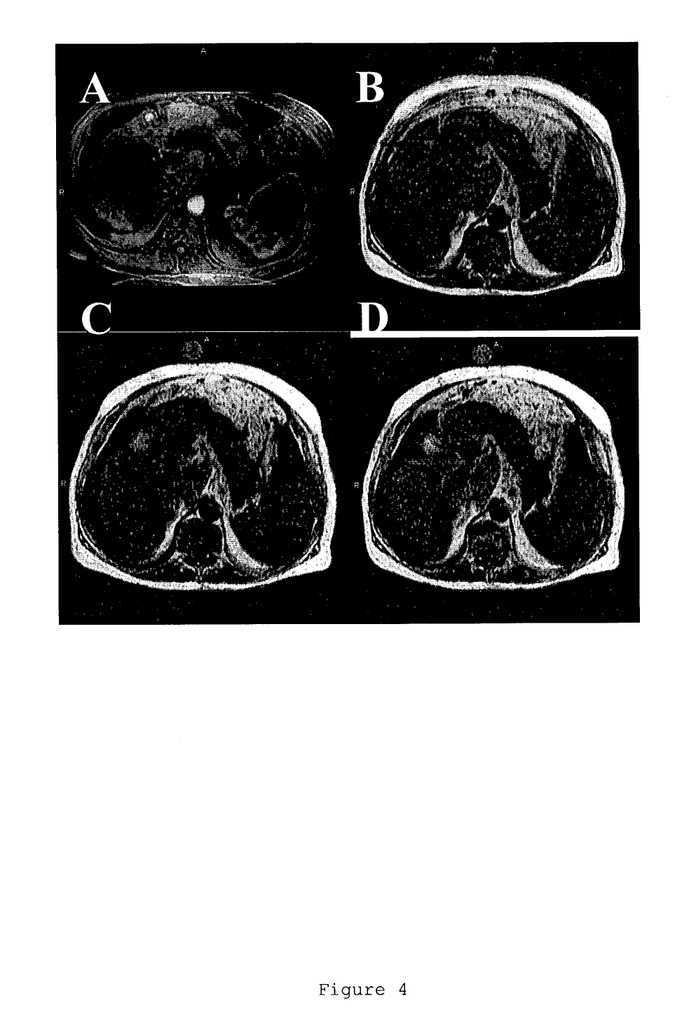

Patient 1: FIG. 4 shows a T1-weighted image obtained during the arterial phase of gadolinium enhancement in a patient with cirrhosis and hepatocellular carcinoma. There is a 1.4 cm hypervascular lesion in the medial segment of the left lobe of the liver (arrow) representing a recurrent lesion. This patient returned a few days after diagnostic MRI for percutaneous chemical ablation therapy. FIG. 4-A is a water-suppressed image of the acetic acid resonance obtained at baseline, prior to injection. Linear signal loss from the needle is seen (arrowhead). Images obtained immediately after the slow, hand injection of approximately half the volume (3 mL) show a focal accumulation of acetic acid within the liver at the site of the lesion (FIGS. 4-B and 4-C). Subsequent images obtained after the injection of a total volume of 6 mL acetic acid demonstrate accumulation of the agent at the needle tip location (FIGS. 4-D and 4-E) . The later images demonstrate a higher signal at the site of injection, suggesting a higher acetic acid concentration in a focal area adjacent to the needle tip. No extra-hepatic extravasation of the agent is observed.

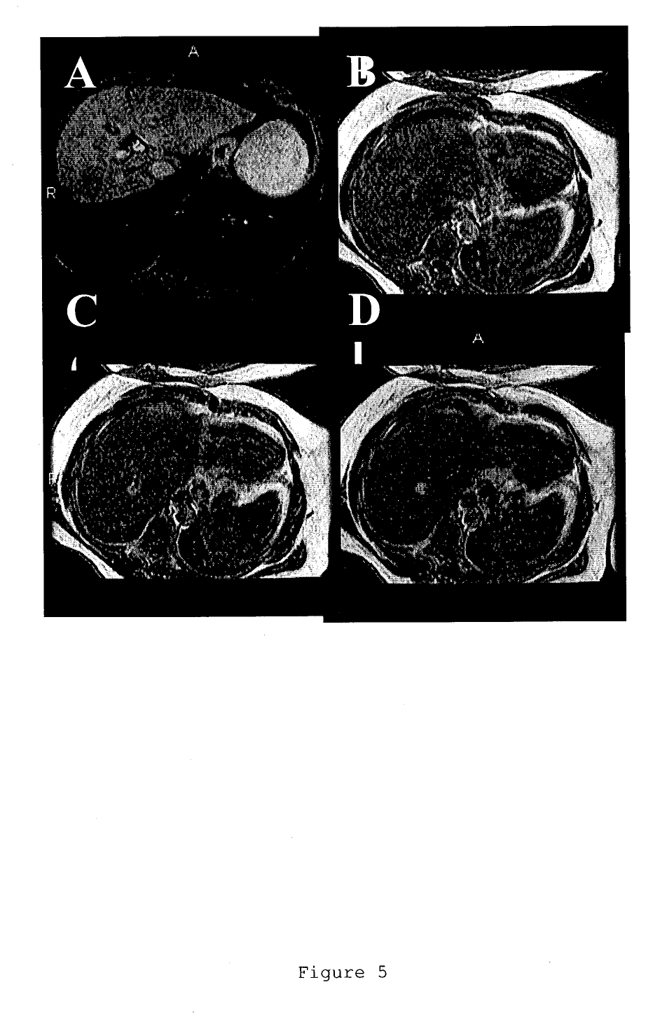

Patient 2: Pre- and post-injection chemical shift MRI was also obtained using identical technique in a different patient with a small hepatocellular carcinoma in the posterior dome (FIG. 5-A). Following acetic acid injection, there is focal high signal at the location of the needle tip (FIG. 5-B) which persists after removal of the needle, thereby confirming that it is not an artifact from metal (FIGS. 5-C and 5-D). The size of the lesion closely matches that of the distribution of acetic acid.

This data demonstrates the effectiveness of Applicants' methods in mapping the intrahepatic acetic acid distribution dynamically during percutaneous chemical ablation therapy. These methods employ a simple pulse sequence that is widely available on most commercial MRI systems. No research modification of the MRI system was necessary to obtain these images. These results indicate that images may be obtained with both high spatial and temporal resolution, allowing for the accurate three-dimensional mapping of acetic acid during percutaneous chemical ablation procedures.

### Example 4

### In Vivo Imaging of Ethanol Injection

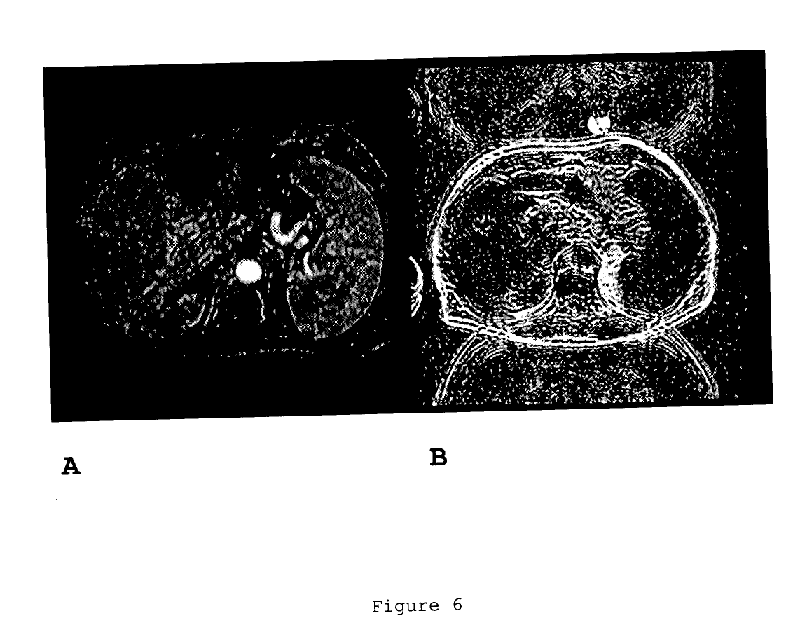

Patient 3: Pre-injection and post-injection chemical shift MRI was obtained during absolute ethanol ablation in a patient with multifocal hepatoma. FIG. 6 illustrates the use of selective suppression CS-MRI to provide guidance during the ethanol chemical ablation procedure. In this case, selective suppression CS-MRI was used. Although the resulting signal from ethanol is low in intensity, focal signal accumulation due to ethanol injection is observed (thick arrow).

### Example 5

Imaging of ¹³C-labeled Acetic Acid Injection

A patient presents for ³C-labeled acetic acid ablation therapy of a hepatic tumor. The patient is prepared by placement on the MRI scanner platform and the spectrometer is calibrated for ¹³C-labeled acetic acid using standard T1 and T2-weighted imaging. An MR-compatible needle is advanced into the lesions to introduce the ablation agent. Dynamic two-dimensional, chemical shift MRI is performed during the slow injection of ¹³C-labeled acetic acid. The distribution of ¹³C-labeled acetic acid is then directly visualized using chemical shift MRI. The patient undergoes the procedure well, without damage to surrounding tissues, and the chemical ablation therapy is successful in destroying the tumor.

### Example 6

### Imaging of ¹³C-labeled Ethanol Injection

A patient presents for ethanol ablation therapy of a hepatic tumor. The patient is prepared by placement on the MRI scanner platform and the spectrometer is calibrated for ¹³C-labeled ethanol using standard T1 and T2-weighted imaging. An MR-compatible needle is advanced into the lesions to introduce the ablation agent. Dynamic two-dimensional, chemical shift MRI is performed during the slow injection of ¹³C-labeled ethanol. The distribution of ¹³C-labeled ethanol is then directly visualized using chemical shift MRI. The patient undergoes the procedure well, without damage to surrounding tissues, and the chemical ablation therapy is successful in destroying the tumor.

### Example 7

### Imaging of Hot ²³Na-Labeled Saline Injection

A patient presents for hot saline ablation therapy of a hepatic tumor. The patient is prepared by placement on the MRI scanner platform and the spectrometer is calibrated for ²³Na-labeled saline using standard T1 and T2-weighted imaging. An MR-compatible needle is advanced into the lesions to introduce the ablation agent. Dynamic two-dimensional, chemical shift MRI is performed during the slow injection of ²³Na-labeled saline. The distribution of ²³Na-labeled saline is then directly visualized using chemical shift MRI. The patient undergoes the procedure well, without damage to surrounding tissues, and the chemical ablation therapy is successful in destroying the tumor.

### Example 8

### Treatment of Prostate Tumors

A patient presents for acetic acid ablation therapy of a prostate tumor. The patient is prepared by placement on the MRI scanner platform and the spectrometer is calibrated for acetic acid using standard T1 and T2-weighted imaging. An MR-compatible needle is advanced into the lesions to introduce the ablation agent. Dynamic two-dimensional, chemical shift MRI is performed during the slow injection of acetic acid. The distribution of acetic acid is then directly visualized using chemical shift MRI. The patient undergoes the procedure well, without damage to surrounding tissues, and the chemical ablation therapy is successful in destroying the tumor.

### Example 9

### Treatment of Lung Tumors

A patient presents for acetic acid ablation therapy of a lung tumor. The patient is prepared by placement on the MRI scanner platform and the spectrometer is calibrated for acetic acid using standard T1 and T2-weighted imaging. An MR-compatible needle is advanced into the lesions to introduce the ablation agent. Dynamic two-dimensional, chemical shift MRI is performed during the slow injection of acetic acid. The distribution of acetic acid is then directly visualized using chemical shift MRI. The patient undergoes the procedure well, without damage to surrounding tissues, and the chemical ablation therapy is successful in destroying the tumor.

### Example 10

### Treatment of Breast Tumors

A patient presents for acetic acid ablation therapy of a breast tumor. The patient is prepared by placement on the MRI scanner platform and the spectrometer is calibrated for acetic acid using standard T1 and T2-weighted imaging. An MR-compatible needle is advanced into the lesions to introduce the ablation agent. Dynamic two-dimensional, chemical shift MRI is performed during the slow injection of acetic acid. The distribution of acetic acid is then directly visualized using chemical shift MRI. The patient undergoes the procedure well, without damage to surrounding tissues, and the chemical ablation therapy is successful in destroying the tumor.

### Example 11

### Treatment of Kidney Tumors

A patient presents for acetic acid ablation therapy of a renal tumor. The patient is prepared by placement on the MRI scanner platform and the spectrometer is calibrated for acetic acid using standard T1 and T2-weighted imaging. An MR-compatible needle is advanced into the lesions to introduce the ablation agent. Dynamic two-dimensional, chemical shift MRI is performed during the slow injection of acetic acid. The distribution of acetic acid is then directly visualized using chemical shift MRI. The patient undergoes the procedure well, without damage to surrounding tissues, and the chemical ablation therapy is successful in destroying the tumor.

The invention being thus described, it will be obvious that the same may be modified or varied in many ways. Such modifications and variations are not to be regarded as a departure from the spirit and scope of the invention and all such modifications and variations are intended to be included within the scope of the following claims.

We claim:

1. A method for mapping distribution of a therapeutic composition in the tissue of an animal, which comprises the steps of:
(i) administering said therapeutic composition to the tissue of said animal, and
(ii) directly visualizing the distribution of said therapeutic composition by chemical shift magnetic resonance imaging attuned to detect said therapeutic composition.

2. The method of claim 1, wherein said therapeutic composition is selected from the group consisting of acetic acid, ethanol, ¹³C-labeled acetic acid, ¹³C-labeled ethanol, and ²³Na-labeled saline.

3. The method of claim 1, wherein said therapeutic composition is acetic acid.

4. The method of claim 1, wherein said magnetic resonance imaging utilizes acetic acid as a contrast agent and is attuned to detect acetic acid resonance.

5. The method of claim 1, wherein said direct visualization is made during administration of said therapeutic composition in a chemical ablation therapy procedure.

6. The method of claim 1, wherein said administration is made by injecting said therapeutic composition into said animal through one or more injection site(s).

7. The method of claim 1, wherein said tissue is a tumor.

8. The method of claim 7, wherein said tumor is a hepatic tumor, a renal tumor, a prostate tumor, a lung tumor, or a breast tumor.

9. The method of claim 1, wherein said method additionally comprises a first step of injecting said tissue with a contrast agent before administering said therapeutic composition and before visualizing the distribution of said therapeutic composition.

10. The method of claim 1, wherein said contrast agent is a gadolinium-containing contrast agent.

11. The method of claim 1, wherein said step of directly visualizing the distribution of said therapeutic composition additionally comprises scanning over a range of chemical shift magnetic resonances attuned to different concentrations of said therapeutic composition.

12. A method for mapping distribution of an acetic acid composition during a chemical ablation therapy procedure in a tumor in an animal, which comprises the steps of:
(i) injecting said acetic acid composition into said tumor in an animal, and
(ii) directly visualizing the distribution of said composition by chemical shift magnetic resonance imaging attuned to detect acetic acid resonance.

13. The method of claim 12, wherein said method additionally comprises a first step of injecting said tissue with a contrast agent before administering said therapeutic composition and before visualizing the distribution of said therapeutic composition.

14. The method of claim 13, wherein said contrast agent is a gadolinium-containing contrast agent.

15. The method of claim 12, wherein said step of directly visualizing the distribution of said composition additionally comprises scanning over a range of chemical shift magnetic resonances attuned to different concentrations of said composition.

16. A method for treating an animal in need of chemical ablation therapy in a target tissue, comprising the steps of:
(i) injecting a therapeutic composition into said target tissue of said animal; and
(ii) directly visualizing the distribution of said therapeutic composition using chemical shift magnetic resonance imaging attuned to detect the resonance of said therapeutic composition.

17. The method of claim 16, comprising the additional step of selecting the volume of said therapeutic composition to be injected, and optionally selecting one or more additional target tissue injection site(s), based on the distribution of said therapeutic composition visualized.

18. The method of claim 16, wherein said therapeutic composition is acetic acid.

19. The method of claim 16, wherein said target tissue is a tumor.

20. The method of claim 19, wherein said tumor is a hepatic tumor, a renal tumor, a prostate tumor, a lung tumor, or a breast tumor.

21. The method of claim 16, wherein said method additionally comprises a first step of injecting said tissue with a contrast agent before administering said therapeutic composition and before visualizing the distribution of said therapeutic composition.

22. The method of claim 21, wherein said contrast agent is a gadolinium-containing contrast agent.

23. The method of claim 16, wherein said step of directly visualizing the distribution of said therapeutic composition additionally comprises scanning over a range of chemical shift magnetic resonances attuned to different concentrations of said therapeutic composition.
